(19) United States
(12) Patent Application Publication
(10) Pub. No.: US 2024/0132462 A1
(43) Pub. Date: Apr. 25, 2024

(54) METHOD FOR PREPARING TERT-BUTYL N-((1R,2S,5S)-2-((2-((5-CHLOROPYRIDIN-2-YL)AMINO)-2-OXOACETYL)AMINO)-5-(DIMETHYLCARBAMOYL)CYCLOHEXYL)CARBAMATE

(71) Applicants: FMC Corporation, Philadelphia, PA (US); FMC Agro Singapore Pte. Ltd., Singapore (SG)

(72) Inventors: Yanchun CAO, Shanghai (CN); Xin LIU, Shanghai (CN); Ning XU, Shanghai (CN); Hao WANG, Shanghai (CN); Zhijian XU, Shanghai (CN)

(21) Appl. No.: 18/274,379

(22) PCT Filed: Jan. 27, 2022
(86) PCT No.: PCT/US22/14035

§ 371 Date: Jul. 26, 2023

Related U.S. Application Data
(60) Provisional application No. 63/143,282, filed on Jan. 29, 2021.

(51) Int. Cl. C07D 401/04

(52) U.S. Cl. CPC C07D 401/04

(57) ABSTRACT

Described herein are novel methods of synthesizing 5-Bromo-2-(3-chloro-pyridin-2-yl)-2H-pyrazole-3-carboxylic acid from pyrazole or pyrazole derivatives.

### CROSS-REFERENCE TO RELATED APPLICATIONS

This application claims the benefit of U.S. Provisional Application No. 63/143,282 filed Jan. 29, 2021.

### FIELD OF INVENTION

This disclosure is directed to novel methods of synthesizing 5-Bromo-2-(3-chloro-pyridin-2-yl)-2H-pyrazole-3-carboxylic acid. Compounds prepared by the methods disclosed herein are useful for preparation of certain anthranilamide compounds that are of interest as insecticides, such as, for example, the insecticides chlorantraniliprole and cyantraniliprole.

### BACKGROUND

Conventional processes for the production of 5-Bromo-2-(3-chloro-pyridin-2-yl)-2H-pyrazole-3-carboxylic acid are subject to several industrial concerns, such as processability, environmental hazards, high cost, reagent reactivity, and necessary specialized equipment.

The present disclosure provides novel methods useful for preparing 5-Bromo-2-(3-chloro-pyridin-2-yl)-2H-pyrazole-3-carboxylic acid and derivatives thereof. The benefits of the methods of the present disclosure compared to previous methods are numerous and include improved overall yield, reduced cost, eliminated need for mixed solvent separations, reduced waste, simplified operation complexity, and reduced process hazards.

### BRIEF DESCRIPTION

In one aspect, provided herein is a method of preparing a compound of Formula VI, wherein

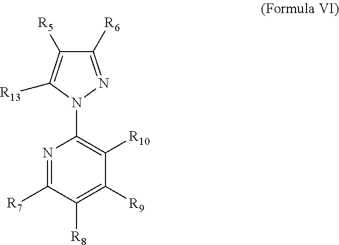

each of R₅-R₁₀ is independently selected from hydrogen and halogen; and
R₁₃ is an organic acid, the method comprising
   I) forming a mixture comprising
      A) a compound of Formula V, wherein

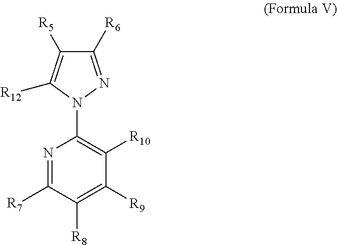

each of R₅-R₁₀ is independently selected from hydrogen and halogen;
R₁₂ is nitrile; and
wherein the compound of Formula V is prepared according to a method comprising
   i) forming a mixture comprising
   a) a compound of Formula III, wherein

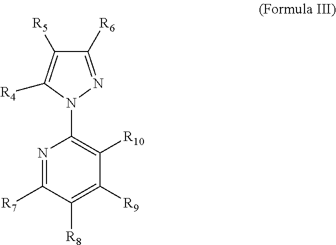

each of R₄-R₁₀ is independently selected from hydrogen and halogen; wherein at least one of R₄, R₅, and R₆ is halogen, and wherein the compound of Formula III is prepared according to a method comprising
IA) forming a mixture comprising
AA) a compound of Formula II, wherein

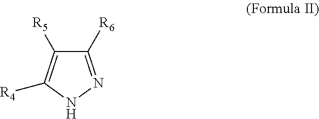

each of R₄, R₅, and R₆ is independently selected from hydrogen and halogen; and
wherein at least one of R₄, R₅, and R₆ is halogen;
BB) a compound of Formula IV, wherein

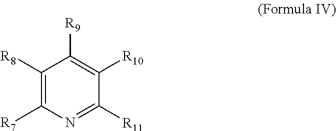

each of R₇-R₁₁ is independently selected from hydrogen and halogen and at least one of R₇-R₁₁ is fluoride, wherein the compound of Formula IV is prepared according to a method comprising
ia) forming a mixture comprising
aa) a compound of Formula VII, wherein

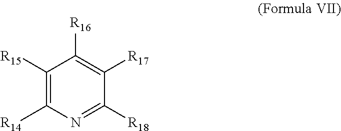

each of R₁₄-R₁₈ is independently selected from hydrogen and halogen; and
none of R₁₄-R₁₈ are fluoride;
bb) a fluoride source;
cc) a solvent; and
dd) optionally a phase catalyst; and
iia) reacting the mixture;
CC) a solvent; and
DD) a base; and
IIA) reacting the mixture;
b) a cyanide reagent;
c) a solvent;
d) a compound comprising a metal; and
e) optionally an additive; and
ii) reacting the mixture; and
B) an acid, base or enzyme; and
II) reacting the mixture.

In one aspect, provided herein is a method of preparing a compound of Formula V, wherein

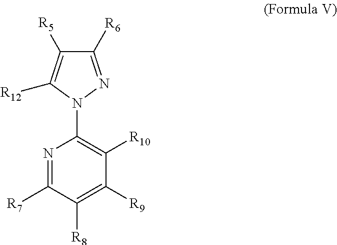

each of R₅-R₁₀ is independently selected from hydrogen and halogen; and
R₁₂ is nitrile, the method comprising
   i) forming a mixture comprising
      a) a compound of Formula III, wherein

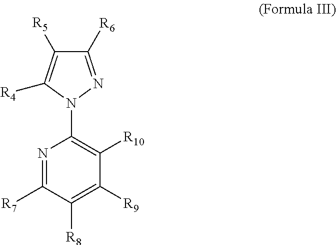

each of R₄-R₁₀ is independently selected from hydrogen and halogen; wherein at least one of R₄, R₅, and R₆ is halogen, and wherein the compound of Formula III is prepared according to a method comprising
IA) forming a mixture comprising
AA) a compound of Formula II, wherein

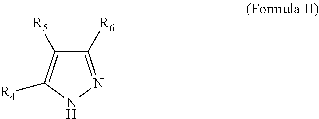

each of R₄, R₅, and R₆ is independently selected from hydrogen and halogen; and
wherein at least one of R₄, R₅, and R₆ is halogen;
BB) a compound of Formula IV, wherein

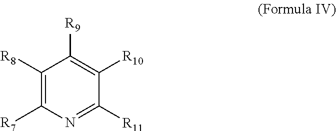

each of R₇-R₁₁ is independently selected from hydrogen and halogen and at least one of R₇-R₁₁ is fluoride, wherein the compound of Formula IV is prepared according to a method comprising
ia) forming a mixture comprising
aa) a compound of Formula VII, wherein

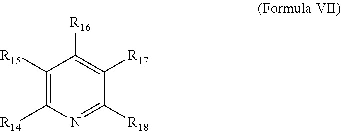

each of R₁₄-R₁₈ is independently selected from hydrogen and halogen; and
wherein at least one of R₁₄-R₁₈ is halogen;
none of R₁₄-R₁₈ are fluoride;
bb) a fluoride source;
cc) a solvent; and
dd) optionally a phase catalyst; and
iia) reacting the mixture;
CC) a solvent; and
DD) a base; and
IIA) reacting the mixture;
b) a cyanide reagent;
c) a solvent;
d) a compound comprising a metal; and
e) optionally an additive; and
ii) reacting the mixture.

In one aspect, provided herein is a method of preparing a compound of Formula III, wherein

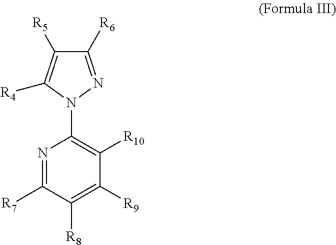

each of R₄-R₁₀ is independently selected from hydrogen and halogen; wherein at least one of R₄, R₅, and R₆ is halogen, the method comprising
   IA) forming a mixture comprising
   AA) a compound of Formula II, wherein

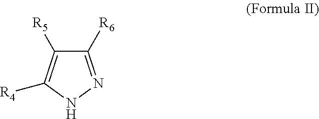

each of R₄, R₅, and R₆ is independently selected from hydrogen and halogen; and
wherein at least one of R₄, R₅, and R₆ is halogen;
BB) a compound of Formula IV, wherein

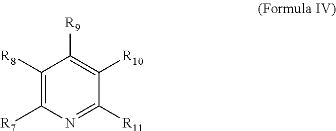

each of R₇-R₁₁ is independently selected from hydrogen and halogen and at least one of R₇-R₁₁ is fluoride, wherein the compound of Formula IV is prepared according to a method comprising
   ia) forming a mixture comprising
      aa) a compound of Formula VII, wherein

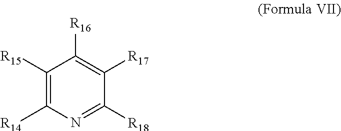

each of R₁₄-R₁₈ is independently selected from hydrogen and halogen; and
wherein at least one of R14-R18 is halogen;
none of R₁₄-R₁₈ are fluoride;
bb) a fluoride source;
cc) a solvent; and
dd) optionally a phase catalyst; and
iia) reacting the mixture;
CC) a solvent; and
DD) a base; and
IIA) reacting the mixture.

### DETAILED DESCRIPTION OF THE DISCLOSURE

As used herein, the terms “comprises,” “comprising,” “includes,” “including,” “has,” “having,” “contains”, “containing,” “characterized by” or any other variation thereof, are intended to cover a non-exclusive inclusion, subject to any limitation explicitly indicated. For example, a composition, mixture, process or method that comprises a list of elements is not necessarily limited to only those elements but may include other elements not expressly listed or inherent to such composition, mixture, process or method.

The transitional phrase “consisting of” excludes any element, step, or ingredient not specified. If in the claim, such would close the claim to the inclusion of materials other than those recited except for impurities ordinarily associated therewith. When the phrase “consisting of” appears in a clause of the body of a claim, rather than immediately following the preamble, it limits only the element set forth in that clause; other elements are not excluded from the claim as a whole.

The transitional phrase “consisting essentially of” is used to define a composition or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

Where an invention or a portion thereof is defined with an open-ended term such as “comprising,” it should be readily understood that (unless otherwise stated) the description should be interpreted to also describe such an invention using the terms “consisting essentially of” or “consisting of.”

Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, the indefinite articles “a” and “an” preceding an element or component of the invention are intended to be nonrestrictive regarding the number of instances (i.e. occurrences) of the element or component. Therefore “a” or “an” should be read to include one or at least one, and the singular word form of the element or component also includes the plural unless the number is obviously meant to be singular.

As used herein, the term “about” means plus or minus 10% of the value.

The term “halogen”, either alone or in compound words such as “haloalkyl”, includes fluorine, chlorine, bromine or iodine. Further, when used in compound words such as “haloalkyl”, said alkyl may be partially or fully substituted with halogen atoms which may be the same or different.

When a group contains a substituent which can be hydrogen, for example R⁴, then, when this substituent is taken as hydrogen, it is recognized that this is equivalent to said group being unsubstituted.

The term “organic base” includes, without limitation, amine compounds (e.g., primary, secondary and tertiary amines), heterocycles including nitrogen-containing heterocycles, and ammonium hydroxide.

The term “inorganic base” includes, without limitation, inorganic compounds with the ability to react with, or neutralize, acids to form salts, such as, for example, metal salts of hydroxide, carbonate, bicarbonate and phosphate.

The term “halogenation reagent” includes, without limitation, halogens and inorganic compounds, such as, for example, bromine, NBS, and 1,3-dibromo-5,5-dimethylhylhydantoin.

The term “phase transfer catalyst” includes compounds that facilitate the migration of a reactant from one phase into another phase where a reaction occurs. Phase transfer catalysis refers to the acceleration of the reaction upon the addition of the phase transfer catalyst.

The term “ether” includes, without limitation, a functional group comprising an ether bond (C—O—C).

The term “nitrile” includes, without limitation, a functional group comprising a nitrile bond (—C≡N).

The term “carboxylic acid” includes, without limitation, a functional group comprising a carboxylic acid bond (C(═O)—OH).

The term “organic acid” includes, without limitation, a functional group that confers acidity and consists of atoms selected from carbon, nitrogen, oxygen, and hydrogen.

Certain compounds of this invention can exist as one or more stereoisomers. The various stereoisomers include enantiomers, diastereomers, atropisomers and geometric isomers. One skilled in the art will appreciate that one stereoisomer may be more active and/or may exhibit beneficial effects when enriched relative to the other stereoisomer(s) or when separated from the other stereoisomer(s). Additionally, the skilled artisan knows how to separate, enrich, and/or to selectively prepare said stereoisomers.

The embodiments of this disclosure include:

Embodiment 1. A method of preparing a compound of Formula VI, wherein

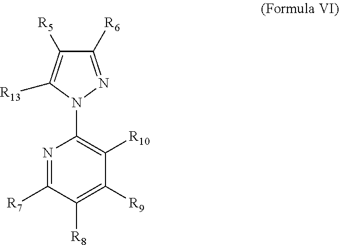

each of R₁₃ is independently selected from hydrogen and halogen; and
R₁₃ is an organic acid, the method comprising
   I) forming a mixture comprising
      A) a compound of Formula V, wherein

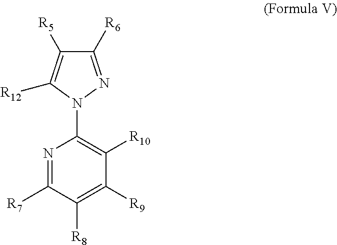

each of R₅-R₁₀ is independently selected from hydrogen and halogen;
R₁₂ is nitrile; and
wherein the compound of Formula V is prepared according to a method comprising
   i) forming a mixture comprising
   a) a compound of Formula III, wherein

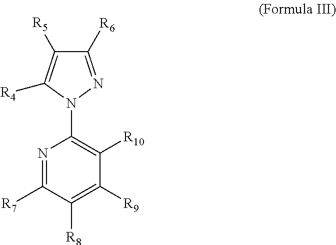

each of R₄-R₁₀ is independently selected from hydrogen and halogen; wherein at least one of R₄, R₅, and R₆ is halogen, and wherein the compound of Formula III is prepared according to a method comprising
IA) forming a mixture comprising
AA) a compound of Formula II, wherein

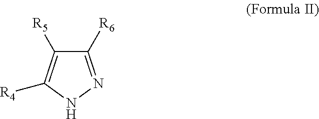

each of R₄, R₅, and R₆ is independently selected from hydrogen and halogen; and
wherein at least one of R₄, R₅, and R₆ is halogen;
BB) a compound of Formula IV, wherein

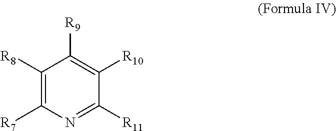

each of R₇-R₁₁ is independently selected from hydrogen and halogen and at least one of R₇-R₁₁ is fluoride, wherein the compound of Formula IV is prepared according to a method comprising
ia) forming a mixture comprising
aa) a compound of Formula VII, wherein

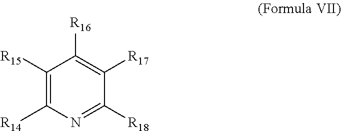

each of R₁₄-R₁₈ is independently selected from hydrogen and halogen; and
wherein at least one of R₁₄-R₁₈ is halogen;
none of R₁₄-R₁₈ are fluoride;
bb) a fluoride source;
cc) a solvent; and
dd) optionally a phase catalyst; and
iia) reacting the mixture;
CC) a solvent; and
DD) a base; and
IIA) reacting the mixture;
b) a cyanide reagent;
c) a solvent;
d) a compound comprising a metal; and
e) optionally an additive; and
ii) reacting the mixture; and
B) an acid, base or enzyme; and
II) reacting the mixture.

Embodiment 2. The method of embodiment 1, wherein the acid is selected from H2SO4, hydrochloric acid (HCl), hydrobromic acid (HBr), formic acid (HCOOH), acetic acid (AcOH) and methyl sulfonic acid(MSA), and combinations thereof; the base is selected from sodium hydroxide, potassium hydroxide, sodium carbonate, potassium carbonate, potassium phosphate, potassium bicarbonate, combinations thereof; and the enzyme is selected from nitrilase, amidohydrolase, combinations thereof.

Embodiment 3. The method of embodiment 1, wherein the method step II) of reacting the mixture occurs at a reaction temperature in the range of about 50° C. to about 120° C.

Embodiment 4. The method of embodiment 1, wherein the compound comprising a metal is a transition metal catalyst.

Embodiment 5. The method of embodiment 1, wherein the cyanide reagent is selected from sodium cyanide, potassium cyanide, copper(I) cyanide, zinc cyanide, potassium hexacyanoferrate(II) and combinations thereof.

Embodiment 6. The method of embodiment 1, wherein the solvent c) is selected from sulfolane, diglyme, triglyme, acetonitrile, toluene,N,N-Dimethylformamide (DMF), N,N-Dimethylacetamide (DMAc), N-methyl-2-pyrrolidone (NMP), and combinations thereof.

Embodiment 7. The method of embodiment 1, wherein the method step ii) of reacting the mixture occurs at a reaction temperature in the range of about 100° C. to about 200° C.

Embodiment 8. The method of embodiment 1, wherein R₅ and R₆ of Formula III are each independently hydrogen.

Embodiment 9. The method of embodiment 1, wherein the inorganic base is selected from sodium hydroxide, potassium hydroxide, potassium carbonate, sodium carbonate, potassium phosphate, lithium hydroxide, lithium carbonate, and organic base is selected from triethylamine, DBU, 1,4-Diazabicyclo[2.2.2]octane(DABCO), sodium methoxide, potassium t-butoxide, potassium methoxide, sodium t-butoxide and combinations thereof.

Embodiment 10. The method of embodiment 1, wherein the solvent CC) is selected from sulfolane, diglyme, triglyme, toluene, N,N-Dimethylformamide (DMF), N,N-Dimethylacetamide (DMAc), N-methyl-2-pyrrolidone (NMP), heavy aromatics s150, heavy aromatics s200 and combinations thereof.

Embodiment 11. The method of embodiment 1, wherein the method step IIA) of reacting the mixture occurs at a reaction temperature in the range of about 100° C. to about 155° C.

Embodiment 12. The method of embodiment 1, wherein the fluoride source is selected from KF, HF, NaF, ZnF 2 and combinations thereof.

Embodiment 13. The method of embodiment 1, wherein the solvent cc) is selected from sulfolane, triglyme, N,N-Dimethylformamide (DMF), N,N-Dimethylacetamide (DMAc), diglyme, N-methyl-2-pyrrolidone (NMP), and combinations thereof.

Embodiment 14. The method of embodiment 1, wherein the phase catalyst is selected from tetramethyl ammonium chloride (TMAC), tetramethylammonium bromide (TMAB), tetramethylammonium iodide (TMAI), tetrabutyl ammonium chloride (TBAC), tetrabutyl ammonium bromide (TBAB), tetrabutyl ammonium iodide (TBAI), aliquat-336, 18-crown-6, 15-crown-5, and combinations thereof.

Embodiment 15. The method of embodiment 1, wherein the method step iia) of reacting the mixture occurs at a reaction temperature in the range of about 100° C. to about 200° C.

Embodiment 16. The method of embodiment 1, wherein the compound of Formula II is prepared according to a method comprising
I) forming a mixture comprising
A) a compound of Formula I, wherein

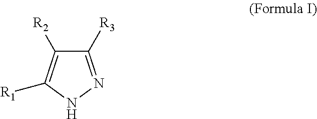

each of R₁, R₂, and R₃ is independently a halogen;
B) a dehalogenation reagent; and
D) a solvent; and
II) reacting the mixture.

Embodiment 17. The method of embodiment 16, wherein the solvent is selected from acetic acid, water, toluene, N,N-dimethylformamide(DMF), N,N-dimethylacetamide(DMAc), 1,3-Dimethyl-2-imidazolidinone(DMI), N-methyl-2-pyrrolidone (NMP), N-methylmorpholine (NMM), diglyme, triglyme, sulfolane, and combinations thereof.

Embodiment 18. The method of embodiment 16, wherein the dehalogenation reagent is selected from sodium sulfite, sodium bisulfite, sodium hyposulfite, sodium thiosulfate, sodium hydrosulfide, sodium sulfate, and combinations thereof.

Embodiment 19. The method of embodiment 16, wherein the additive is selected from sodium iodide, iodine, potassium iodide, tetra-n-butyl ammonium iodide, and combinations thereof.

Embodiment 20. The method of embodiment 16, wherein the method step II) of reacting the mixture occurs at a reaction temperature in the range of about 100° C. to about 180° C.

Embodiment 21. The method of embodiment 16, wherein the compound of Formula I is prepared according to a method comprising
I) forming a mixture comprising
A) pyrazole or a pyrazole derivative;
B) a halogenation reagent;
   C) a reaction solvent comprising water and optionally an organic solvent; and
D) optionally an inorganic base; and
II) reacting the mixture.

Embodiment 22. The method of embodiment 21, wherein the halogenation reagent comprises
A) a reagent selected from hydrogen bromide, bromine, N-bromosuccinimide, 1,3-dibromo-5,5-dimethylhylhydantoin, sodium bromide, potassium bromide, and combinations thereof; and
B) optionally hydrogen peroxide.

Embodiment 23. The method of embodiment 21, wherein the base is selected from sodium hydroxide, potassium hydroxide, potassium carbonate, sodium carbonate, potassium phosphate, lithium hydroxide, sodium methoxide, lithium carbonate, sodium acetate, potassium acetate, and combinations thereof.

Embodiment 24. The method of embodiment 21, wherein the method step II) of reacting the mixture occurs at a reaction temperature in the range of about −10° C. to about 70° C.

Embodiment 25. A method of preparing a compound of Formula V, wherein

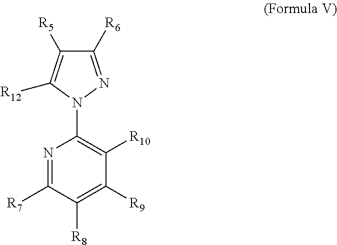

each of R₅-R₁₀ is independently selected from hydrogen and halogen; and
R₁₂ is nitrile, the method comprising
   i) forming a mixture comprising
      a) a compound of Formula III, wherein

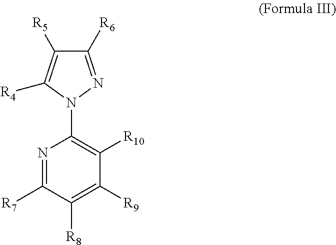

each of R₄-R₁₀ is independently selected from hydrogen and halogen; wherein at least one of R₄, R₅, and R₆ is halogen, and wherein the compound of Formula III is prepared according to a method comprising
   IA) forming a mixture comprising
   AA) a compound of Formula II, wherein

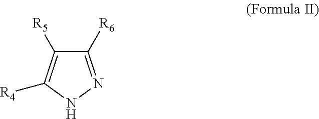

each of R₄, R₅, and R₆ is independently selected from hydrogen and halogen; and
wherein at least one of R₄, R₅, and R₆ is halogen;
BB) a compound of Formula IV, wherein

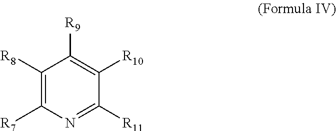

each of R₇-R₁₁ is independently selected from hydrogen and halogen and at least one of R7-R₁₁ is fluoride, wherein the compound of Formula IV is prepared according to a method comprising
ia) forming a mixture comprising
aa) a compound of Formula VII, wherein

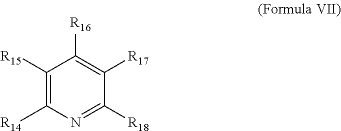

each of R₁₄-R₁₈ is independently selected from hydrovgen and halogen; and
wherein at least one of R₁₄-R₁₈ is halogen;
none of R₁₄-R₁₈ are fluoride;
∠bb) a fluoride source;
cc) a solvent; and
dd) optionally a phase catalyst; and
iia) reacting the mixture;
CC) a solvent; and
DD) abase; and
IIA) reacting the mixture;
b) a cyanide reagent;
c) a solvent;
d) a compound comprising a metal; and
ii) reacting the mixture.

Embodiment 26. The method of embodiment 25, wherein the compound comprising a metal is a transition metal catalyst.

Embodiment 27. The method of embodiment 25, wherein the cyanide reagent is selected from sodium cyanide, copper(I) cyanide, zinc cyanide, potassium cyanide, potassium hexacyanoferrate(II) and combinations thereof.

Embodiment 28. The method of embodiment 25, wherein the solvent c) is selected from sulfolane, diglyme, triglyme, acetonitrile, toluene,N,N-Dimethylformamide (DMF), N,N-Dimethylacetamide (DMAc), N-methyl-2-pyrrolidone (NMP), and combinations thereof.

Embodiment 29. The method of embodiment 25, wherein the method step ii) of reacting the mixture occurs at a reaction temperature in the range of about 100° C. to about 200° C.

Embodiment 30. The method of embodiment 25, wherein R₅ and R₆ of Formula III are each independently hydrogen.

Embodiment 31. The method of embodiment 25, wherein the inorganic base is selected from sodium hydroxide, potassium hydroxide, potassium carbonate, sodium carbonate, potassium phosphate, lithium hydroxide, lithium carbonate, and organic base is selected from triethylamine, DBU, 1,4-Diazabicyclo[2.2.2]octane(DABCO), sodium methoxide, potassium t-butoxide, potassium methoxide, sodium t-butoxide and combinations thereof.

Embodiment 32. The method of embodiment 25, wherein the solvent CC) is selected from sulfolane, diglyme, triglyme, toluene, N,N-Dimethylformamide (DMF), N,N-Dimethylacetamide (DMAc), N-methyl-2-pyrrolidone (NMP), heavy aromatics s150, heavy aromatics s200 and combinations thereof.

Embodiment 33. The method of embodiment 25, wherein the method step IIA) of reacting the mixture occurs at a reaction temperature in the range of about 100° C. to about 200° C.

Embodiment 34. The method of embodiment 25, wherein the fluoride source is selected from KF, HF, NaF, ZnF₂ and combinations thereof.

Embodiment 35. The method of embodiment 25, wherein the solvent cc) is selected from sulfolane, N,N-Dimethylformamide (DMF), N,N-Dimethylacetamide (DMAc), diglyme, triglyme, N-methyl-2-pyrrolidone (NMP), and combinations thereof.

Embodiment 36. The method of embodiment 25, wherein the phase catalyst is selected from tetramethyl ammonium chloride (TMAC), tetramethylammonium bromide (TMAB), tetramethylammonium iodide (TMAI), tetrabutyl ammonium chloride (TBAC), tetrabutyl ammonium bromide (TBAB), tetrabutyl ammonium iodide (TBAI), aliquat-336, 18-crown-6,15-crown-5 and combinations thereof.

Embodiment 37. The method of embodiment 25, wherein the method step iia) of reacting the mixture occurs at a reaction temperature in the range of about 100° C. to about 200° C.

Embodiment 38. The method of embodiment 25, wherein the compound of Formula II is prepared according to a method comprising
I) forming a mixture comprising
A) a compound of Formula I, wherein

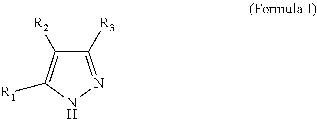

each of R_{,}R₂, and R₃ is independently a halogen;
B) a dehalogenation reagent;
D) a solvent; and
II) reacting the mixture.

Embodiment 39. The method of embodiment 38, wherein the solvent is selected from acetic acid, water, toluene, N,N-dimethylformamide(DMF), N,N-dimethylacetamide(DMAc), 1,3-Dimethyl-2-imidazolidinone(DMI), N-methyl-2-pyrrolidone (NMP), N-methylmorpholine (NMM), diglyme, triglyme, sulfolane, and combinations thereof.

Embodiment 40. The method of embodiment 38, wherein the dehalogenation reagent is selected from sodium sulfite, sodium bisulfite, sodium hyposulfite, sodium thiosulfate, sodium hydrosulfide, sodium sulfate, and combinations thereof.

Embodiment 41. The method of embodiment 38, wherein the additive is selected from sodium iodide, iodine, potassium iodide, tetra-n-butyl ammonium iodide, and combinations thereof.

Embodiment 42. The method of embodiment 38, wherein the method step II) of reacting the mixture occurs at a reaction temperature in the range of about 100° C. to about 180° C.

Embodiment 43. The method of embodiment 38, wherein the compound of Formula I is prepared according to a method comprising
I) forming a mixture comprising
A) pyrazole or a pyrazole derivative;
B) a halogenation reagent;
   C) a reaction solvent comprising water and optionally an organic solvent; and
D) optionally a base; and
II) reacting the mixture.

Embodiment 44. The method of embodiment 43, wherein the halogenation reagent comprises
A) a reagent selected from hydrogen bromide, bromine, N-bromosuccinimide, 1,3-dibromo-5,5-dimethylhylhydantoin, sodium bromide, potassium bromide, and combinations thereof; and
B) optionally hydrogen peroxide.

Embodiment 45. The method of embodiment 43, wherein the inorganic base is selected from sodium hydroxide, potassium hydroxide, potassium carbonate, sodium carbonate, potassium phosphate, lithium hydroxide, sodium methoxide, lithium carbonate, sodium acetate, potassium acetate, and combinations thereof.

Embodiment 46. The method of embodiment 43, wherein the method step II) of reacting the mixture occurs at a reaction temperature in the range of about −10° C. to about 70° C.

Embodiment 47. A method of preparing a compound of Formula III, wherein

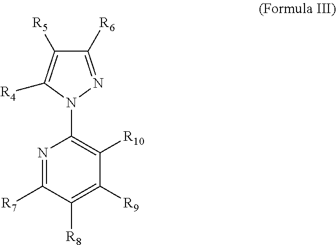

each of R₄-R₁₀ is independently selected from hydrogen and halogen; wherein at least one of R₄, R₅, and R₆ is halogen, the method comprising
   IA) forming a mixture comprising
      AA) a compound of Formula II, wherein

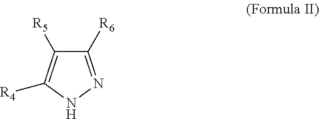

each of R₄, R₅, and R₆ is independently selected from hydrogen and halogen; and
wherein at least one of R₄, R₅, and R₆ is halogen;
BB) a compound of Formula IV, wherein

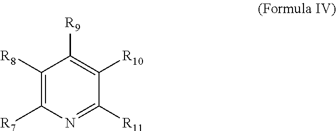

each of R₇-R₁₁ is independently selected from hydrogen and halogen and at least one of R₇-R₁₁ is fluoride, wherein the compound of Formula IV is prepared according to a method comprising
   ia) forming a mixture comprising
   aa) a compound of Formula VII, wherein

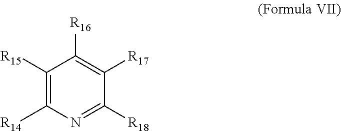

each of R₁₄-R₁₈ is independently selected from hydrogen and halogen; and
wherein at least one of R₁₄-R₁₈ is halogen, none of R₁₄-R₁₈ are fluoride;
bb) a fluoride source;
cc) a solvent; and
dd) optionally a phase catalyst; and
iia) reacting the mixture;
CC) a solvent; and
DD) a base; and
IIA) reacting the mixture.

Embodiment 48. The method of embodiment 47, wherein R₅ and R₆ of Formula III are each independently hydrogen.

Embodiment 49. The method of embodiment 47, wherein the inorganic base is selected from sodium hydroxide, potassium hydroxide, potassium carbonate, sodium carbonate, potassium phosphate, lithium hydroxide, lithium carbonate, and organic base is selected from triethylamine, DBU, 1,4-Diazabicyclo[2.2.2]octane(DABCO), sodium methoxide, potassium t-butoxide, potassium methoxide, sodium t-butoxide and combinations thereof.

Embodiment 50. The method of embodiment 47, wherein the solvent CC) is selected from sulfolane, diglyme, triglyme, toluene, N,N-Dimethylformamide (DMF), N,N-Dimethylacetamide (DMAc), N-methyl-2-pyrrolidone (NMP), heavy aromatics s150, heavy aromatics s200 and combinations thereof.

Embodiment 51. The method of embodiment 47, wherein the method step IIA) of reacting the mixture occurs at a reaction temperature in the range of about 100° C. to about 155° C.

Embodiment 52. The method of embodiment 47, wherein the fluoride source is selected from KF, HF, NaF, ZnF₂ and combinations thereof.

Embodiment 53. The method of embodiment 47, wherein the solvent cc) is selected from sulfolane,triglyme, N,N-Dimethylformamide (DMF), N,N-Dimethylacetamide (DMAc), diglyme, N-methyl-2-pyrrolidone (NMP), and combinations thereof.

Embodiment 54. The method of embodiment 47, wherein the phase catalyst is selected from tetramethyl ammonium chloride (TMAC), tetramethylammonium bromide (TMAB), tetramethylammonium iodide (TMAI), tetrabutyl ammonium chloride (TBAC), tetrabutyl ammonium bromide (TBAB), aliquat-336, 18-crown-6, 15-crown-5 and combinations thereof.

Embodiment 55. The method of embodiment 47, wherein the method step iia) of reacting the mixture occurs at a reaction temperature in the range of about 100° C. to about 200° C.

Embodiment 56. The method of embodiment 47, wherein the compound of Formula II is prepared according to a method comprising
I) forming a mixture comprising
A) a compound of Formula I, wherein

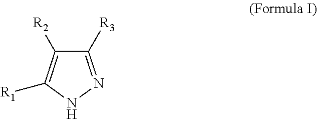

each of R₁, R₂, and R₃ is independently a halogen;
B) a dehalogenation reagent;
C) additive,
D) a solvent; and
II) reacting the mixture.

Embodiment 57. The method of embodiment 56, wherein the solvent is selected from acetic acid, water, toluene, N,N-dimethylformamide(DMF), N,N-dimethylacetamide(DMAc), 1,3-Dimethyl-2-imidazolidinone(DMI), N-methyl-2-pyrrolidone (NMP), N-methylmorpholine (NMM), diglyme, triglyme, sulfolane, and combinations thereof.

Embodiment 58. The method of embodiment 56, wherein the dehalogenation reagent is selected from sodium sulfite, sodium bisulfite, sodium hyposulfite, sodium thiosulfate, sodium hydrosulfide, sodium sulfate, and combinations thereof.

Embodiment 59. The method of embodiment 56, wherein the additive is selected from sodium iodide, iodine, potassium iodide, tetra-n-butyl ammonium iodide, and combinations thereof.

Embodiment 60. The method of embodiment 56, wherein the method step II) of reacting the mixture occurs at a reaction temperature in the range of about 100° C. to about 180° C.

Embodiment 61. The method of embodiment 56, wherein the compound of Formula I is prepared according to a method comprising
I) forming a mixture comprising
A) pyrazole or a pyrazole derivative;
B) a halogenation reagent;
   C) a reaction solvent comprising water and optionally an organic solvent;
D) optionally a base; and
II) reacting the mixture.

Embodiment 62. The method of embodiment 61, wherein the halogenation reagent comprises
A) a reagent selected from hydrogen bromide, bromine, N-bromosuccinimide, 1,3-dibromo-5,5-dimethylhylhydantoin, sodium bromide, potassium bromide, and combinations thereof; and
B) optionally hydrogen peroxide.

Embodiment 63. The method of embodiment 61, wherein the inorganic base is selected from sodium hydroxide, potassium hydroxide, potassium carbonate, sodium carbonate, potassium phosphate, lithium hydroxide, sodium methoxide, lithium carbonate, sodium acetate, potassium acetate, and combinations thereof.

Embodiment 64. The method of embodiment 61, wherein the method step II) of reacting the mixture occurs at a reaction temperature in the range of about −10° C. to about 70° C.

In one aspect, a compound of Formula VI is prepared according to a method represented by Scheme 1. The R groups are as defined anywhere in this disclosure.

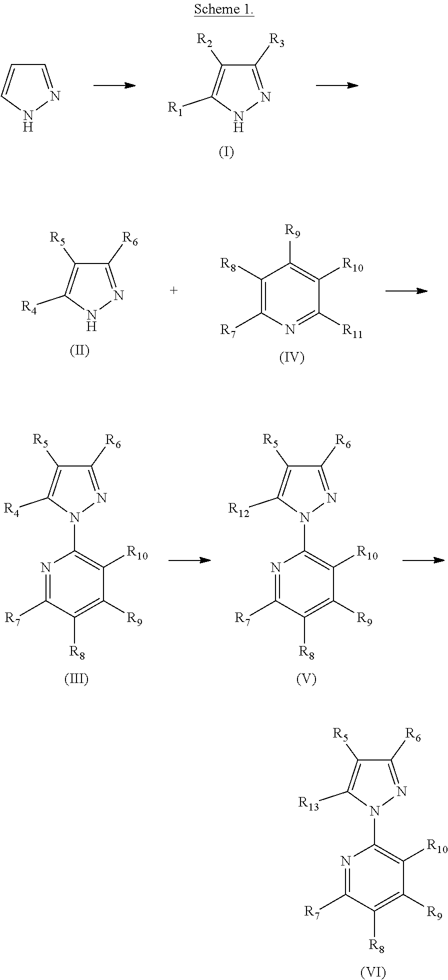

In one aspect, 5-Bromo-2-(3-chloro-pyridin-2-yl)-2H-pyrazole-3-carboxylic acid is prepared according to a method represented by Scheme 2.

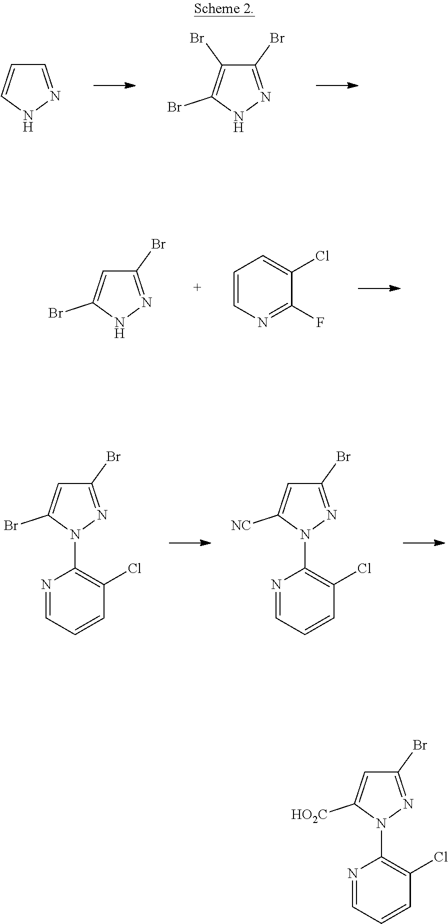

In one aspect, a compound of Formula I is prepared according to a method represented by Scheme 3. The R groups are as defined anywhere in this disclosure.

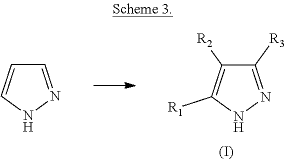

This aspect includes reacting pyrazole with a halogenation reagent in a reaction solvent including water and optionally an organic solvent, and optionally in the presence of an inorganic base. In one embodiment, the halogenation reagent is selected from hydrogen peroxide/HBr, Bromine (Br₂), N-bromosuccinimide, 1,3-dibromo-5,5-dimethylhylhydantoin hydrogen peroxide/NaBr, hydrogen peroxide/KBr, hydrogen peroxide/Br₂, and combinations thereof. In another embodiment, the halogenation reagent is Br₂. In one embodiment, inorganic base is selected from sodium hydroxide, potassium hydroxide, sodium acetate, potassium acetate and combinations thereof. In another embodiment the inorganic base is power sodium hydroxide. In one embodiment, the reaction temperature is in the range from about −10° C. to about 70° C. In another embodiment, the reaction temperature is in the range from about 0° C. to about 20° C. In one embodiment, the organic solvent is selected from tert-Butyl methyl ether (MBTE), dichloromethane (DCM), dichloroethane (DCE), chloroform, diethyl ether and combinations thereof. In another embodiment, the organic solvent is MTBE.

In one aspect, a compound of Formula II is prepared according to a method represented by Scheme 4. The R groups are as defined anywhere in this disclosure.

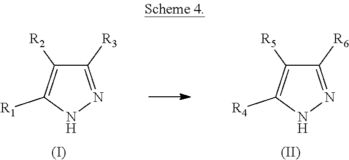

This aspect includes reacting a compound of Formula I with a dehalogenation reagent in a solvent in the presence of a reducing agent. In one embodiment, the solvent is selected from acetic acid, water, N,N-dimethylformamide (DMF), N,N-dimethylacetamide (DMAc), diglyme, triglyme, sulfolane, 1,3-Dimethyl-2-imidazolidinone(DMI), N-methyl-2-pyrrolidone (NMP), N-methylmorpholine (NMM) and combinations thereof. In another embodiment, the solvent is N,N-dimethylacetamide (DMAc). In one embodiment, the additive is selected from sodium iodide, iodine, potassium iodide, tetra-n-butyl ammonium iodide (TBAI), and combinations thereof. In another embodiment, the additive is potassium iodide. In one embodiment, the dehalogenation reagent is selected from sodium sulfite, sodium bisulfite, sodium hyposulfite, sodium thiosulfate, sodium hydrosulfide, sodium sulphate, and combinations thereof. In another embodiment, the dehalogenation reagent is sodium sulfite. In one embodiment, the reaction temperature is in the range from about 100° C. to about 180° C. In another embodiment, the reaction temperature is in the range from about 120° C. to about 150° C.

In one aspect, a compound of Formula IV is prepared according to a method represented by Scheme 5. The R groups are as defined anywhere in this disclosure.

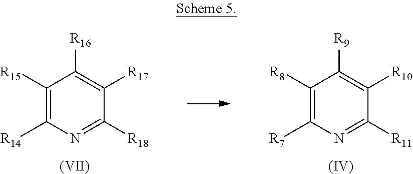

This aspect includes the step of reacting a compound of Formula VII with a halide source in a solvent and optionally in the presence of a phase catalyst. In one embodiment, the halide source is selected from a fluoride source, a chloride source, a bromide source, an iodide source, and combinations thereof. In another embodiment, the halide source is a fluoride source. In one embodiment, the fluoride source is selected from HF, KF, NaF, ZnF₂ and combinations thereof. In another embodiment, the fluoride source is KF. In one embodiment, the phase catalyst is selected from tetramethyl ammonium chloride (TMAC), tetramethylammonium bromide (TMAB), tetramethylammonium iodide (TMAI), tetrabutyl ammonium chloride (TBAC), tetrabutyl ammonium bromide (TBAB), aliquat-336, 18-crown-6, 15-crown-5 and combinations thereof. In another embodiment, the phase catalyst is tetramethyl ammonium chloride (TMAC). In one embodiment, the solvent is selected from sulfolane, N,N-Dimethylformamide (DMF), N,N-Dimethylacetamide (DMAc), diglyme, N-methyl-2-pyrrolidone (NMP), triglyme and combinations thereof. In another embodiment, the solvent is sulfolane. In one embodiment, the reaction temperature is in the range from about 100° C. to about 200° C. In another embodiment, the temperature is in the range from about 165° C. to about 180° C.

In one aspect, a compound of Formula III is prepared according to a method represented by Scheme 6. The R groups are as defined anywhere in this disclosure.

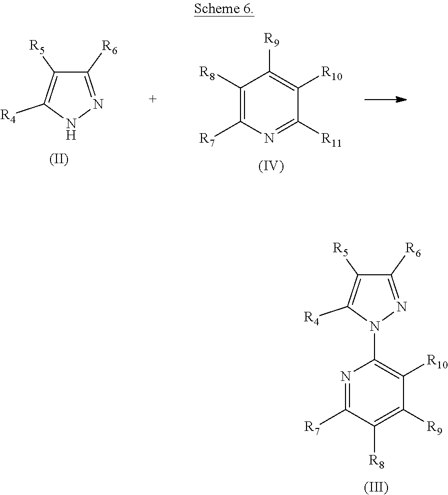

This aspect includes the step of mixing a compound of Formula II with a compound of Formula IV in a solvent in the presence of abase. In one embodiment, the inorganic base is selected from sodium hydroxide, potassium hydroxide, potassium carbonate, sodium carbonate, potassium phosphate, lithium hydroxide, lithium carbonate, and organic base is selected from triethylamine, DBU, 1,4-Diazabicyclo[2.2.2]octane(DABCO), sodium methoxide, potassium t-butoxide, potassium methoxide, sodium t-butoxide and combinations thereof. In another embodiment, the base is potassium carbonate. In one embodiment, the solvent is selected from toluene, N,N-dimethylformamide (DMF), N,N-dimethylacetamide (DMAc), N-methyl-2-pyrrolidone (NMP), diglyme, triglyme, sulfolane heavy aromatics s150, heavy aromatics s200 and combinations thereof. In another embodiment, the solvent is sulfolane. In one embodiment, the reaction temperature ranging is in the range from about 100° C. to about 200° C. In another embodiment, the temperature is in the range from about 110° C. to about 140° C.

In one aspect, a compound of Formula V is prepared according to a method represented by Scheme 7. The R groups are as defined anywhere in this disclosure.

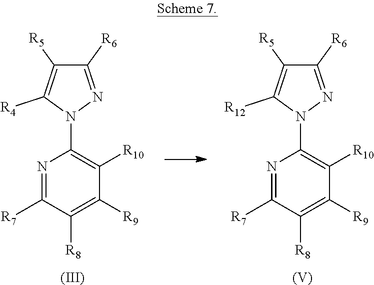

This aspect includes mixing a compound of Formula III with a cyanide reagent in a solvent in the presence of copper salt and optionally an additive. In one embodiment, the solvent is selected from sulfolane, diglyme, triglyme, acetonitrile, toluene, acetonitrile and toluene, N,N-Dimethylformamide (DMF), N,N-Dimethylacetamide (DMAc), N-methyl-2-pyrrolidone (NMP), and combinations thereof. In another embodiment, the solvent is diglyme. In one embodiment, the cyanide reagent is selected from sodium cyanide, copper(I) cyanide, zinc cyanide, potassium cyanide, potassium hexacyanoferrate(II) and combinations thereof. In another embodiment, the cyanide reagent is sodium cyanide. In one embodiment, the copper salt is selected from cuprous iodide, cuprous bromide, cuprous oxide, cuprous chloride, copper acetate and combinations thereof. In another embodiment, the copper salt is cuprous iodide. In another embodiment, the copper salt is cuprous chloride. In one embodiment, the additive is potassium iodide, ethylene glycol, propylene glycol, water, glycerin, glucose, cyclodextrin, sodium iodide, iodine and combinations thereof. In another embodiment, the copper salt is cuprous chloride and the additive is ethylene glycol. In one embodiment, the reaction temperature is in the range from about 100° C. to about 200° C. In another embodiment, the reaction temperature is in the range from about 110° C. to about 150° C.

In one aspect, a compound of Formula VI is prepared according to a method represented by Scheme 8. The R groups are as defined anywhere in this disclosure.

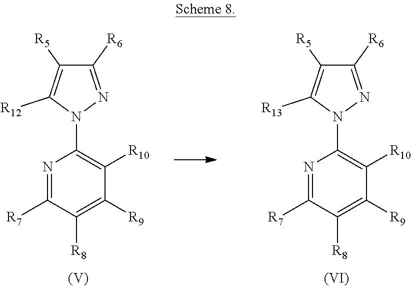

This aspect includes reacting a compound of Formula V in the presence of an acid, a base and enzyme. In one embodiment, the acid is selected from concentrated H2SO4, hydrochloric acid (HCl), hydrobromic acid (HBr), formic acid (HCOOH), acetic acid (AcOH) and methylsulfonic acid (MSA), combinations thereof. In one embodiment, the base is selected from sodium hydroxide, potassium hydroxide, sodium carbonate, potassium carbonate, potassium phosphate, potassium bicarbonate, combinations thereof. In one embodiment, the enzyme is selected from nitrilase, amidohydrolase, combinations thereof. In another embodiment, the acid is H2SO4. In one embodiment, the reaction temperature is in the range from 50° C. to 120° C. In another embodiment, the reaction temperature is in the range from 60° C. to 100° C.

### EXAMPLES

Without further elaboration, it is believed that one skilled in the art using the preceding description can utilize the present invention to its fullest extent. The following Examples are, therefore, to be construed as merely illustrative, and not limiting of the disclosure in any way whatsoever. The starting material for the following Examples may not have necessarily been prepared by a particular preparative run whose procedure is described in other Examples. It also is understood that any numerical range recited herein includes all values from the lower value to the upper value. For example, if a range is stated as 10-50, it is intended that values such as 12-30, 20-40, or 30-50, etc., are expressly enumerated in this specification. These are only examples of what is specifically intended, and all possible combinations of numerical values between and including the lowest value and the highest value enumerated are to be considered to be expressly stated in this application.

### Example 1

### Hydrogen Peroxide/HBr as a Halogenation Reagent

34 grams of pyrazole and 505.8 g of 48% hydrogen bromide solution were charged to a reactor. 170 grams of 30% hydrogen peroxide was added drop-wise at 0° C. over 2 hours. The reaction temperature was controlled at 0-30° C. After reaction, the product was precipitated as a solid, and then the reaction mixture was quenched with 10% sodium sulfite. After filtration and drying, 142 g of high purity (95%, LC Area) of 3,4,5-tribromo-1H-pyrazole was obtained.

### Example 2

### Bromine/Sodium Hydroxide as a Halogenation Reagent

34 grams of pyrazole was dissolved in water and then sodium hydroxide was added at 0° C. to obtain the corresponding pyrazole sodium salt. Next, 239.7 g of bromine was added drop-wise at 0° C. over 2 hours. The reaction temperature was controlled at 20-40° C. After reaction, the product was precipitated as a solid, and then the reaction mixture was quenched with 10% sodium sulfite. After filtration and drying, 147 g of high purity (98%, LC Area) of 3,4,5-tribromo-1H-pyrazole was obtained.

### Example 3

### Potassium Iodide/Sodium Sulfite as a Dehalogenation Reagent

100 grams of 3,4,5-tribromo-1H-pyrazole, 1.1 g of KI, and 62 g of Na₂SO₃ in 300 mL DMAc were reacted at 160-180° C. for 5 hours to completed reaction. After completion of the reaction, the reaction mixture was filtered, and then DMAc was distilled off under vacuum. Next, water was added to the crude product. The reaction mixture was stirred for 10 min. The product, 3,5-dibromo-1H-pyrazole, was precipitated as a solid. After filtration and drying, 68 g of high purity (98%, LC Area) of 3,5-dibromo-1H-pyrazole was obtained.

### Example 4

### Halide Source

300.0 g of 2,3-dichloropyridine, 22.2 g TMAC and 176.7 g KF are reacted in a vessel. The reaction temperature was controlled in the range of 170-175° C. After completion of the reaction, the reaction mass was cooled to 25-30° C. 3-chloro-2-fluoropyridine was distilled off under reduced pressure. After distillation, 267.0 g of high purity (95%, LC Area) of 3-chloro-2-fluoropyridine was obtained, which could be used in subsequent steps.

### Example 5

### Coupling Reaction

50.0 g of 3,5-dibromo-1H-pyrazole, 29.2g 3-chloro-2-fluoropyridine and 36.7 g potassium carbonate were reacted in a vessel. The reaction temperature was controlled at 120-125° C. After completion of the reaction, water and MTBE were introduced to the reaction mass at 25-30° C. The reaction mass was stirred for 15 minutes and then the reaction mass was separated into two layers. The organic layer was concentrated at 40-45° C. under reduced pressure to obtain crude 3-chloro-2-(3,5-dibromo-1H-pyrazol-1-yl)pyridine. After concentration, 81.0 g of high purity (87.5%, wt %) of 3-chloro-2-(3,5-dibromo-1H-pyrazol-1-yl)pyridine was obtained

### Example 6-1

### Reaction in the Presence of a Transition Metal Catalyst

0.95 grams of CuI, 3.32 g KI, and 5.4 g NaCN were added to a solution of 33.8 g 3-chloro-2-(3,5-dibromo-1H-pyrazol-1-yl)pyridine in DMAc at 10-25° C. Next, the reaction mixture was stirred at 130-140° C. for 6 hours to complete reaction. DMAc was distilled off under vacuum. Toluene was added and stirred for 30 minutes. Next, the solution was filtered and toluene was removed under vacuum. After filtration and drying, 23.2 g (94%, LC Area) of 3-bromo-1-(3 -chloropyridin-2-yl)-1H-pyrazole-5-carbonitrile was obtained.

### Example 6-2

### Reaction with Cuprous Chloride as the Metal Comprising Compound

0.35 g CuCl, 2.6 g ethylene glycol and 2.5g NaCN were added to a solution of 15.2 g 3-chloro-2-(3,5-dibromo-1H-pyrazol-1-yl)pyridine in diglyme at 20-25° C. Then the reaction mixture was stirred at 115-120° C. for 16 hours to complete reaction. After completion of reaction, charged with 7% NaClO solution (0.2eq.), water and Methyl tert-butyl ether (MTBE) to the reaction mass at 25-30° C., stirred the reaction mass for 15 min, the reaction mass was settled for 15 mins to obtain two separate layers. Organic layer was concentrated at 40-45° C. under reduced pressure then obtained 12.6 g (94%, LC Area) 3-bromo-1-(3-chloropyridin-2-yl)-1H-pyrazole-5-carbonitrile.

### Example 6-3

### Reaction with Cuprous Chloride as the Metal Comprising Compound

0.35 g CuCl and 2.5 g NaCN were added to a solution of 15.2 g 3-chloro-2-(3,5-dibromo-1H-pyrazol-1-yl)pyridine in diglyme at 20-25° C. Then the reaction mixture was stirred at 115-120° C. for 18 hours, and the reaction conversion rate was 50% LCA; Then continue stirring this reaction for another 8 hours, the conversion rate is still 50% LCA. The reaction was stalled.

### Example 7

### Acidification

28.4 grams of high purity (94%, LC Area) 3-bromo-1-(3-chloropyridin-2-yl)-1H-pyrazole-5-carbonitrile was dissolved in 50% H₂SO₄ solution and charged to a flask. The mixture was heated to 80-85° C. and kept at this temperature for 3-4 hours to complete reaction. NaOH solution was used to adjust pH to a value in the range of about 9 to about 10 to precipitate the corresponding 5-bromo-2-(3-chloro-pyridin-2-yl)-2H-pyrazole-3-carboxylic acid sodium salt. H2SO4 was then used to adjust pH to a value in the range of about 1 to about 2 to precipitate 5-bromo-2-(3 -chloro-pyridin-2-yl)-2H-pyrazole-3 -carboxylic acid. After filtration and drying, 28.6 g (98%, LC Area) of 5-bromo-2-(3-chloro-pyridin-2-yl)-2H-pyrazole-3-carboxylic acid was obtained.

This written description uses examples to illustrate the present disclosure, including the best mode, and also to enable any person skilled in the art to practice the disclosure, including making and using any devices or systems and performing any incorporated methods. The patentable scope of the disclosure is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A method of preparing a compound of Formula VI, wherein 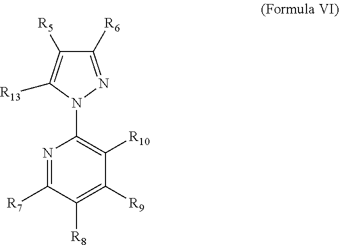
each of R₅-R₁₀ is independently selected from hydrogen and halogen; and
R₁₃ is an organic acid, the method comprising
I) forming a mixture comprising
A) a compound of Formula V, wherein 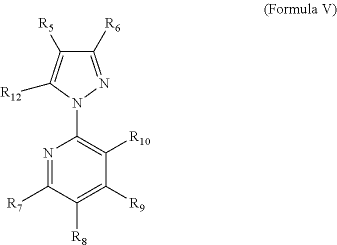
each of R₅-R₁₀ is independently selected from hydrogen and halogen;
R₁₂ is nitrile; and
wherein the compound of Formula V is prepared according to a method comprising
i) forming a mixture comprising
a) a compound of Formula III, wherein 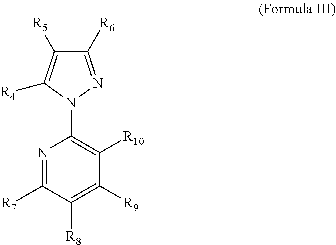
each of R₄-R₁₀ is independently selected from hydrogen and halogen; wherein at least one of R₄, R₅, and R₆ is halogen, and wherein the compound of Formula III is prepared according to a method comprising
IA) forming a mixture comprising
AA) a compound of Formula II, wherein 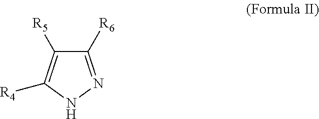
each of R₄, R₅, and R₆ is independently selected from hydrogen and halogen; and
wherein at least one of R₄, R₅, and R₆ is halogen;
BB) a compound of Formula IV, wherein 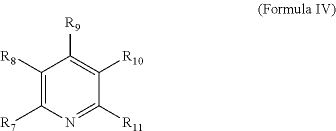
each of R₇-R₁₁ is independently selected from hydrogen and halogen and at least one of R₇-R₁₁ is fluoride, wherein the compound of Formula IV is prepared according to a method comprising
ia) forming a mixture comprising
aa) a compound of Formula VII, wherein 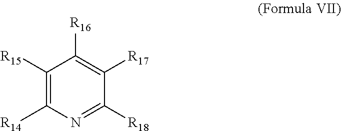
each of R₁₄-R₁₈ is independently selected from hydrogen and halogen, wherein at least one of R₁₄-R₁₈ is halogen; and
none of R₁₄-R₁₈ are fluoride;
bb) a fluoride source;
cc) a solvent; and
dd) optionally a phase transfer catalyst; and
iia) reacting the mixture;
CC) a solvent; and
DD) a base; and
IIA) reacting the mixture;
b) a cyanide reagent;
c) a solvent;
d) a compound comprising a metal; and
e) optionally an additive; and
ii) reacting the mixture; and
B) an acid, base or enzyme; and
II) reacting the mixture.

2. The method of claim 1, wherein the acid is selected from H₂SO₄, hydrochloric acid (HCl), hydrobromic acid (HBr), formic acid (HCOOH), acetic acid (AcOH) and methylsulfonic acid (MSA), and combinations thereof; the base is selected from sodium hydroxide, potassium hydroxide, sodium carbonate, potassium carbonate, potassium phosphate, potassium bicarbonate, combinations thereof; and the enzyme is selected from nitrilase, amidohydrolase, and combinations thereof.

3. The method of claim 1, wherein the method step II) of reacting the mixture occurs at a reaction temperature in the range of about 50° C. to about 120° C.

4. The method of claim 1, wherein the compound comprising a metal is a transition metal catalyst.

5. The method of claim 1, wherein the transition metal catalyst is selected from cuprous iodide, cuprous bromide and cuprous chloride, Copper(I)oxide, cuprous triflate (CuOTf), Copper(I) acetate and combinations thereof.

6. The method of claim 1, wherein the additive e) is selected from ethylene glycol, propylene glycol, water, glycerin, glucose, cyclodextrin, potassium iodide, sodium iodide, iodine and combinations thereof.

7. The method of claim 1, wherein the compound comprising metal d) is cuprous chloride, and the additive e) is ethylene glycol.

8. The method of claim 1, wherein the cyanide reagent is selected from sodium cyanide, potassium cyanide, copper(I) cyanide, zinc cyanide, potassium hexacyanoferrate (II) and combinations thereof.

9. The method of claim 1, wherein the solvent c) is selected from sulfolane, diglyme, triglyme, acetonitrile, toluene, N,N-Dimethylformamide (DMF), N,N-Dimethylacetamide (DMAc), N-methyl-2-pyrrolidone (NMP), and combinations thereof.

10. A method of preparing a compound of Formula V, wherein 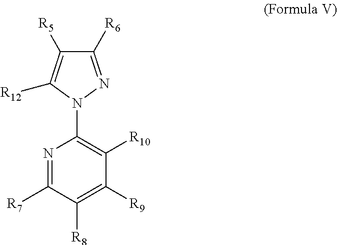
each of R₅-R₁₀ is independently selected from hydrogen and halogen; and
R₁₂ is nitrile, the method comprising
i) forming a mixture comprising
a) a compound of Formula III, wherein 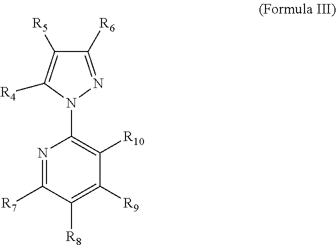
each of R₄-R₁₀ is independently selected from hydrogen and halogen; wherein at least one of R₄, R₅, and R₆ is halogen, and wherein the compound of Formula III is prepared according to a method comprising
IA) forming a mixture comprising
AA) a compound of Formula II, wherein 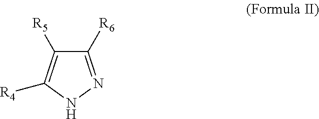
each of R₄, R₅, and R₆ is independently selected from hydrogen and halogen; and
wherein at least one of R₄, R₅, and R₆ is halogen;
BB) a compound of Formula IV, wherein 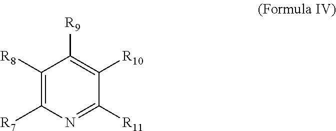
each of R₇-R₁₁ is independently selected from hydrogen and halogen and at least one of R₇-R₁₁ is fluoride, wherein the compound of Formula IV is prepared according to a method comprising
ia) forming a mixture comprising
aa) a compound of Formula VII, wherein 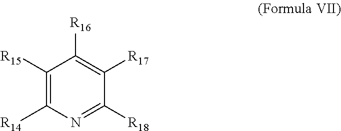
each of R₁₄-R₁₈ is independently selected from hydrogen and halogen; and
wherein at least one of R₁₄-R₁₈ is halogen, none of R₁₄-R₁₈ are fluoride;
bb) a fluoride source;
cc) a solvent; and
dd) optionally a phase transfer catalyst; and
iia) reacting the mixture;
CC) a solvent; and
DD) a base; and
IIA) reacting the mixture;
b) a cyanide reagent;
c) a solvent;
d) a compound comprising a metal; and
e) optionally an additive; and
ii) reacting the mixture.

11. The method of claim 10, wherein the compound comprising a metal is a transition metal catalyst.

12. The method of claim 10, wherein the transition metal catalyst is selected from cuprous iodide, cuprous bromide and cuprous chloride, Copper(I)oxide, cuprous triflate (CuOTf), Copper(I) acetate and combinations thereof.

13. The method of claim 10, wherein the additive e) is selected from ethylene glycol, propylene glycol, water, glycerin, glucose, cyclodextrin, potassium iodide, sodium iodide, iodine and combinations thereof.

14. The method of claim 10, wherein the compound comprising metal d) is cuprous chloride, and the additive e) is ethylene glycol.

15. The method of claim 10, wherein the cyanide reagent is selected from sodium cyanide, copper(I) cyanide, zinc cyanide, potassium cyanide, potassium hexacyanoferrate(II) and combinations thereof.

16. The method of claim 10, wherein the solvent c) is selected from sulfolane, diglyme, triglyme, acetonitrile, toluene, N,N-Dimethylformamide (DMF), N,N-Dimethylacetamide (DMAc), N-methyl-2-pyrrolidone (NMP), and combinations thereof.

17. A method of preparing a compound of Formula III, wherein 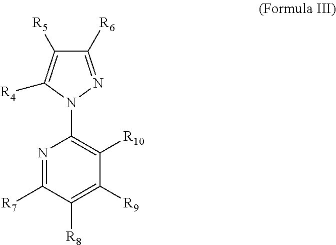
each of R₄-R₁₈ is independently selected from hydrogen and halogen; wherein at least one of R₄, R₅, and R₆ is halogen, the method comprising
IA) forming a mixture comprising
AA) a compound of Formula II, wherein 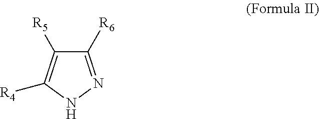
each of R₄, R₅, and R₆ is independently selected from hydrogen and halogen; and
wherein at least one of R₄, R₅, and R₆ is halogen;
BB) a compound of Formula IV, wherein 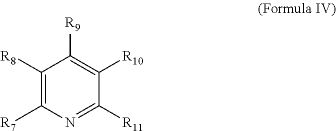
each of R₇-R₁₁ is independently selected from hydrogen and halogen and at least one of R₇-R₁₁ is fluoride, wherein the compound of Formula IV is prepared according to a method comprising
ia) forming a mixture comprising
aa) a compound of Formula VII, wherein 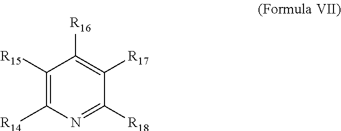
each of R₁₄-R₁₈ is independently selected from hydrogen and halogen; and
wherein at least one of R₁₄-R₁₈ is halogen,
none of R₁₄-R₁₈ are fluoride;
bb) a fluoride source;
cc) a solvent; and
dd) optionally a phase transfer catalyst; and
iia) reacting the mixture;
CC) a solvent; and
DD) a base; and
IIA) reacting the mixture.

18. The method of claim 17, wherein R₅-R₆ of Formula III are each independently hydrogen.

19. The method of claim 17, wherein the inorganic base is selected from sodium hydroxide, potassium hydroxide, potassium carbonate, sodium carbonate, potassium phosphate, lithium hydroxide, lithium carbonate, and organic base is selected from triethylamine, DBU, 1,4-Diazabicyclo[2.2.2]octane(DABCO), sodium methoxide, potassium t-butoxide, potassium methoxide, sodium t-butoxideand combinations thereof.

20. The method of claim 17, wherein the solvent CC) is selected from sulfolane, diglyme, triglyme, toluene, N,N-Dimethylformamide (DMF), N,N-Dimethylacetamide (DMAc), N-methyl-2-pyrrolidone (NMP), and combinations thereof.
